(19) United States
(12) Patent Application Publication
(10) Pub. No.: US 2006/0057628 A1
(43) Pub. Date: Mar. 16, 2006

(54) Method for prognosticating the progress of breast cancer and compounds useful for prevention or treatment thereof

(76) Inventors: Pirkko Vihko, Helsinki (FI); Veli Isomaa, Kempele (FI)

Correspondence Address: YOUNG & THOMPSON, 745 SOUTH 23RD STREET, 2ND FLOOR, ARLINGTON, VA, 22202, US

(21) Appl. No.: 11/225,095

(22) Filed: Sep. 14, 2005

Related U.S. Application Data
(63) Continuation-in-part of application No. PCT/FI04/00145, filed on Mar. 3, 2004.

(30) Foreign Application Priority Data
Mar. 14, 2003 (FI) 20030393

(51) Int. Cl. C12Q 1/68; G01N 33/574; A61K 31/55; C07D 498/14

(52) U.S. Cl. 435/6; 435/7.23; 514/214.01; 540/578

(57) ABSTRACT

This invention concerns in vitro method for prognosticating the progress of breast cancer, comprising detecting or quantifying the level of 17β-hydroxysteroid dehydrogenase (17HSD) type 1 enzyme in breast tumor tissue sample, wherein the presence of said 17HSD type 1 enzyme is indicative of severe progress of breast cancer. Furthermore, the invention also concerns the use of a 17HSD type 1 enzyme inhibitor for prevention or treatment of breast cancer. This invention concerns also the use of compound A in the manufacture of a pharmaceutically acceptable preparation useful as 17HSD inhibitor.

### FIELD OF THE INVENTION

This invention relates to a method for prognosticating the progress of breast cancer, based on the expression of 17β-hydroxysteroid dehydrogenase type 1 enzyme in the breast cancer tissue. The invention also concerns a method for the prevention or treatment of breast cancer by administering an effective amount of a 17β-hydroxysteroid dehydrogenase type 1 enzyme inhibitor to an individual. Further, this invention relates also to the use of compounds known per se as 17β-hydroxysteroid dehydrogenase type 1 inhibitors, and to pharmaceutical preparations comprising said compounds.

### BACKGROUND OF THE INVENTION

The publications and other material are used herein to illuminate the background of the invention and they are incorporated herein by reference.

Enzymes possessing 17β-hydroxysteroid dehydrogenase (17HSD) activity have been cloned and described in the literature. So far at least eight types of such (17HSD types 1 to 8) human enzymes have been discovered. (Peltoketo et al., J Mol Endocrinol (1999) 23, 1-11).

The 17HSD enzymes control the last step in the formation of androgens and estrogens. 17HSD type 1 is a protein of 327 amino acids catalyzing the formation of high-activity 17β-estradiol from low-activity estrone. Type 2 17HSD converts the high-activity 17β-estradiol to the low-activity estrone and high-activity testosterone to low-activity androstenedione and dihydrotestosterone to 5α-androstanedione.

It has been reported that 40% of all cancers, namely breast, prostate, ovarian and uterine cancers are sex steroid sensitive and are thus candidates for approaches based on control of intracrine activity. Therefore it is suggested to treat sex steroid sensitive diseases by specific inhibitors of the 17HSD enzymes.

Molecules able to block the enzyme activity have been screened. Characteristics of such inhibitory molecules, which mainly have a substrate or cofactor-like core structure, have been reported in the literature (Penning Endocrine-Related Cancer (1996) 3, 41-56; Tremblay and Poirier J Chem Soc Perkin Trans 1, (1996), 2765-71; Poirier et al., J Steroid Biochem Molec Biol (1998), 64, 83-90). Tremblay and Poirier describe an estradiol derivative, 16-[carbamoyl(bromomethyl)alkyl]-estradiol, and tested the same in respect of its inhibition of the estradiol formation catalyzed by the enzyme 17HSD type 1. It was found that this compound moderately inhibited the biosynthesis of estradiol. Poirier et al. describe 6β-thiaheptan butyl methyl amide derivative of estradiol as a potent and selective inhibitor of the 17HSD type 1 enzyme. However, the ability of these molecules to inhibit 17HSD type 2 enzyme has not been tested.

There are certain criteria for 17HSD type 1 inhibitors to be used as therapeutic agents. The inhibitor shall be specific for the enzyme in question, i.e. it shall have little or no affinity for other 17HSDs participating in the estrogen metabolism. In particular, it should not possess substantial affinity to 17HSD type 2 enzyme. The inhibitors should not otherwise prevent or modulate steroidogenesis, either. In addition, the inhibitor molecules should lack affinity for the estrogen receptor, thus eliminating any possible agonist effects.

Using in situ hybridization, both 17HSD type 1 and 2 have been detected in benign breast tissue (Miettinen et al., Breast Cancer Res Treat (1999) 57, 175-182). The activities and mRNAs of both enzymes are also present in several breast cancer cell lines (Miettinen et al., Biochem J (1996) 314,839-845). Expression of 17HSD type 1 in about half of breast cancer specimens has been shown using immunohistochemistry (Poutanen et al., Int J Cancer (1992) 50, 266-390).

The growth-promoting influence of 17HSD type 1 has been demonstrated in cultured MCF-7 breast cancer cell line (American Type Culture Collection ATCC) transfected with cDNA of 17HSD type 1 (Miettinen et al., Int J Cancer (1995) 68, 600-604). In nontransfected MCF-7 cells, estradiol induces cell proliferation but estrone does not have any effect on the cell growth. In cells stably transfected with 17HSD type 1, both estradiol and estrone have similar growth-promoting effects showing that 17HSD type 1 is needed to convert estrone to estradiol.

In a review article on 17β-hydroxysteroid dehydrogenases and cancer Vihko et al (Journal of Steroid Biochemistry & Molecular Biology, vol 83(1-5), p. 119-122, 2002) show that both type 1 and 2 17HSD enzymes are present in normal breast and in malignant breast cells. No evidence of the roles of these enzymes in the prognostication of breast cancer is shown.

Gunnarsson et al (Cancer Research, vol 61, p. 8448-8451, 2001) have studied the expression of type 1 and 2 17HSD in two selected patient groups (both 24 patients) by PCR. They found out that the lack of type 2 enzyme combined with high content of type 1 enzyme is a prognostic factor for breast cancer. The role of type 1 enzyme alone as an independent prognostic factor has not been shown.

Gunnarsson et al (Oncogene, vol 22(1), p. 34-40, 2003) have also studied the amplification of type 1 17HSD gene in breast cancer. They state that the amplification of HSD17B1 gene is a prognostic factor in breast cancer. However, the material they used was not statistically significant (p=0.052). Furthermore, the amplification of a gene and the expression thereof are different subjects. It is not self-evident that the levels of genes, mRNAs and proteins correlate with each other.

The use of 17HSD type 1 enzyme in breast tumor has not been suggested as a marker for prognosticating the progress of breast cancer. Moreover, there are no reports on pharmaceutical preparations comprising 17HSD type 1 enzyme inhibitors for use in prevention or treatment of breast cancer.

### SUMMARY OF THE INVENTION

According to one aspect, this invention concerns an in vitro method for prognosticating the progress of breast cancer, comprising detecting or quantifying the level of 17β-hydroxysteroid dehydrogenase (17HSD) type 1 enzyme in breast cancer tissue sample, wherein the presence of said 17HSD type 1 enzyme is independently indicative of severe progress of breast cancer.

According to another aspect, the invention concerns a pharmaceutical composition comprising compound A as disclosed in Figure I, or a pharmaceutically acceptable salt of said compound.

According to further aspect, this invention concerns the use of compound A as disclosed in FIG. 1, or a pharmaceutically acceptable salt of said compound, in the manufacture of a pharmaceutically acceptable preparation useful as 17HSD type 1 inhibitor.

According to still further aspect, this invention concerns the use of said 17HSD type 1 inhibitor, compound A, for prevention or treatment of disorders caused by the 17HSD type 1 enzyme activity, such as human breast cancer. In this respect, postmenopausal women form a special group since 17HSD type 1 is not expressed in the normal breast tissue of postmenopausal patients.

According to still further aspect, this invention concerns a method for prevention or treatment of disorders caused by the 17HSD type 1 enzyme activity, such as human breast cancer, by administering effective amount of compound to a patient suffering from said disorder.

### BRIEF DESCRIPTION OF THE DRAWINGS

FIG. 1 shows the structure of compound A

### DETAILED DESCRIPTION OF THE INVENTION

The inventors of the present invention have carried out a patient study in which cancer tissue specimens from 794 breast cancer patients were analyzed. The expression of 17HSD type 1 and type 2 mRNAs were analyzed in cancer tissue specimens using in situ -hybridization technique as described previously (Oduwole et al., Int J Cancer (2002) 97, 1-6). The immunohistochemical stainings of 17HSD type 1 protein were made using a method described previously (Poutanen et al., Int J Cancer (1992) 50, 266-390).

In the study it was found that breast cancer patients with breast tumors expressing 17HSD type 1 have significantly shorter overall and disease-free survival than all other cases (p=0.0010, 0.0134, log rank). The probability of metastasis formation is higher among these patients.

Type 1 17HSD as well as type 2 are expressed in normal breast tissue in premenopausal women. However, most women having the breast cancer are postmenopausal and were shown not to express type 1 17HSD in their normal breast tissue.

In the breast cancer tissue material from 794 breast cancer patients, multivariate Cox analysis (forward stepwise regression) was used to determine the possible independent prognostic significance of the following parameters: tumor size, the presence of nodal and distant metastases, grade of the tumor, estrogen receptor (ER)α, ERβ, progesterone receptor, 17HSD type 1, type 2, type 5, Ki67 and c-erb-b2. According to the analysis tumor size, 17HSD type 1 and ERα had independent prognostic value (Table 1).

TABLE 1
Multivariate Cox analyses (forward stepwise
regression) for prognostic factors

95% CI for RR

| Step | Factors | Sig | RR | Lower | Upper |
|---|---|---|---|---|---|
| Step | T | <0.001 | 2.424 | 1.676 | 3.504 |
| 1 | 17HSD1 | <0.001 | 4.245 | 1.968 | 9.159 |
| Step | T | <0.001 | 2.734 | 1.843 | 4.056 |
| 2 | 17HSD1 | <0.001 | 4.186 | 1.927 | 9.096 |
| | ERα | <0.013 | 0.408 | 0.201 | 0.829 |

Sig: significance;
RR: relative risk;
CI: confidence interval,
T: size of the tumor,
ER: estrogen receptor

Based on these findings, the inventors suggest that the expression of 17HSD type 1 enzyme in breast cancer tissue is indicative of severe progress of breast cancers. Furthermore this enzyme can be considered as an independent marker for such severe progress of breast cancer. Such independent markers are not previously known in the art. In postmenopausal women it is also a breast cancer marker. The adequate level of said expression is such that it can be detected using standard techniques well known in the art.

The detection or quantification of the 17HSD type 1 enzyme can be performed by any known suitable method. Such methods include for example hybridizing techniques; PCR techniques or immunological methods based on detection of an antibody recognizing the enzyme. The hybridizing techniques include, for example nucleotide hybridization and Northern blot. The detection or quantification of the antibody can be performed according to standard immunoassay protocols, such as label-linked immunosorbent assays, Western blot and immunohistochemical methods. These methods are well known to a person skilled in the art.

Examples of such methods for determining the level or expression of 17HSD type 1 enzyme and other related methods are described in Cancer Research (2004) 65, 7604-7609: “17β-Hydroxysteroid Dehydrogenase Type I Is an Independent Prognostic Marker in Breast Cancer”. This is the corresponding publication by the current inventors and it is incorporated herein by reference.

The inventors of the present invention have surprisingly found a small molecule from a vast amount of compounds from a commercial library (BioSpecs Inc. The Netherlands) which molecule is capable of specifically inhibiting 17HSD type 1 enzyme but not 17HSD type 2 enzyme. The screening of the library compounds was performed using two specific cell lines made by the inventors, which cell lines stably expressed either 17HSD type 1 or 17HSD type 2 enzyme, but not the other one.

For the useful effect of 17HSD type 1 enzyme inhibitor it is essential that the inhibitor is substantially specific for type 1 enzyme and not for type 2 enzyme, since these enzymes have contrary effects. The inhibition of type 1 enzyme will have a positive effect on the treatment of disorders caused by the 17HSD type 1 enzyme activity, such as breast cancer and its progression. On the other hand, the inhibition of type 2 enzyme would have a negative effect when treating such disorders, because it is beneficial to have 17HSD type 2 activity inactivating estradiol. To find such a molecule from a vast amount of compounds is an extreme task and may not have been possible without the specific cell lines mentioned above.

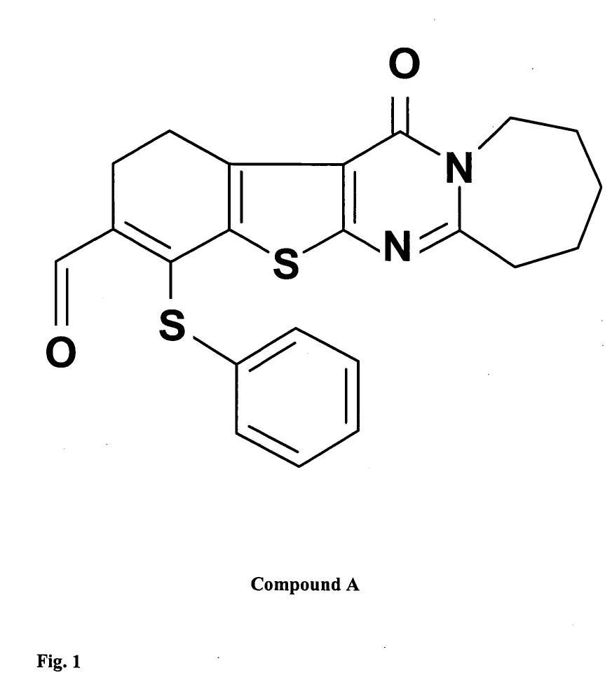

An inhibitor of the invention includes the compound A of FIG. 1, a pharmaceutically acceptable salt of said compound or a derivative of said compound. The expression ‘derivative’ used herein denotes any compound derived using the original compound as the lead compound.

For the purpose of this invention, the 17HSD enzyme inhibitor or its pharmaceutically acceptable salt or derivative can be administered by various routes and as various pharmaceutical forms well known in the art. The suitable administration forms include, for example, oral formulations; topical formulations; parenteral injections including intravenous, intramuscular, intradermal and subcutaneous injections; and transdermal or rectal formulations.

Suitable oral formulations include e.g. tablets, lozenges or capsules prepared by conventional means with pharmaceutically acceptable excipients such as binding agents (e.g. pregelatinized maize starch, polyvinylpyrrolidone or hydroxypropyl methyl cellulose), fillers (e.g. lactose, microcrystalline cellulose or calcium hydrogen phosphate), lubricants (e.g. magnesium stearate, talc or silica), disintegrants (e.g. potato starch or sodium starch glycolate) or wetting agents (e.g. sodium lauryl sulphate). The tablets may be coated by methods well known in the art. Liquid preparations for oral administration may take the form of for example solutions, syrups or suspensions, or they may be presented as a dry product for constitution with water or other suitable vehicle before use. Such liquid preparations may be prepared by conventional means with pharmaceutically acceptable additives such as suspending agents (e.g. sorbitol syrup, cellulose derivatives or hydrogenated edible fats), emulsifying agents (e.g. lecithin or acacia), non-aqueous vehicles (e.g. almond oil, oily esters, ethyl alcohol or fractionated vegetable oils) and preservatives (e.g. methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound.

Suitable formulations for parenteral administration include e.g. bolus injection or continuous infusion. Formulations for injection may be presented in unit dosage form, e.g. in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g. sterile pyrogen-free water, before use.

The required dosage of the 17HSD enzyme type 1 inhibitor will vary with the particular breast cancer being treated or prevented, the severity of the condition, the duration of the treatment, the administration route and the specific compound being employed. Suitable dose ranges may be calculated by those skilled in the art.

The invention will now be illuminated by the following non-restrictive Experimental Section.

Experimental Section

Compound A, which is known per se and the structure of which is shown in FIG. 1, was purchased from BioSpecs Inc. The compound is disclosed in a BioSpecs' database (CD disk September 1998; database SB8I-20T.db; compound A: ID-number AH-262/33341026). The use of this compound as a medicament has not been reported previously.

The tested compounds (about 1000 selected different compounds) were screened in respect of 17HSD enzyme activities according to the following method:

The compounds were screened in respect of 17HSD enzyme activities in vitro on two established MCF-7 cell lines, each stably expressing one 17HSD isoenzyme, either 17HSD type 1 or 17HSD type 2, but not the other one. The interconversion of substrate by each isoenzyme and the HSD-inhibiting activity of chemical compounds in these cell lines was detected by HPLC system (Miettinen et al., Int J Cancer (1996) 68, 600-604).

Varying amounts of the test compounds were incubated in the growth medium of the 17HSD expressing cells together with tritium labeled substrate (estrone for 17HSD type 1 enzyme; 20 nM). The medium samples were removed after exact incubation time and the reaction was stopped by deepfreezing. The samples were analyzed by HPLC-coupled flow scintillation analysis. The 17HSD-inhibiting activity of the test compounds was compared to known reference compound apigenin and to a negative control.

Results:

For one of the most potent 17HSD type 1 inhibitors, Compound A, the inhibition of the 17HSD type 1 enzyme was 80% at a concentration of 1 μM (average of three tests: 81%, 77% and 83%) and 95% at a concentration of 10 μM (average of two tests: 94 and 96%). The reference compound apigenin gave an inhibition activity of 48% at a concentration of 1 μM. The tested compound did not show any inhibition effect on the 17HSD type-2 enzyme (the reference compound apigenin gave a 5% inhibition of the 17HSD type-2 enzyme). Based on this test results, this compound can be considered as a very potent and selective 17HSD-1 inhibitor.

It will be appreciated that the methods of the present invention can be incorporated in the form of a variety of embodiments, only a few of which are disclosed herein. It will be apparent for the expert skilled in the field that other embodiments exist and do not depart from the spirit of the invention. Thus, the described embodiments are illustrative and should not be construed as restrictive.

1. A method for prognosticating the progress of human breast cancer in vitro, comprising detecting or quantifying the level of 17β-hydroxysteroid dehydrogenase (17HSD) type 1 enzyme in breast tumor tissue sample, characterized in that the presence of said 17HSD type 1 enzyme is used to independently indicate the severe progress of breast cancer.

2. The method according to claim 1 characterized in that the 17HSD type 1 enzyme is detected or quantified by nucleotide hybridization, by immunological or immunohistochemical methods or by PCR.

3. Compound A: 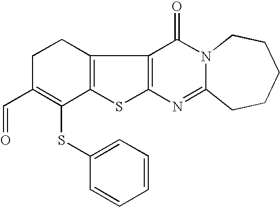
or a pharmaceutically acceptable salt thereof for use as a medicament.

4. Compound A or a pharmaceutically acceptable salt thereof of claim 3 characterized in that said medicament is for prevention or treatment of disorders caused by the 17HSD type 1 enzyme activity.

5. Compound A or a pharmaceutically acceptable salt thereof of claim 3 characterized in that said medicament is for prevention or treatment of human breast cancer.

6. A pharmaceutical composition characterized in comprising compound A: 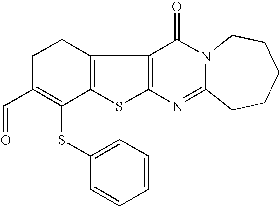
or a pharmaceutically acceptable salt thereof.

7. Pharmaceutical composition of claim 6, characterized in that it is in the form of an oral formulation, injection, transdermal formulation or rectal formulation.

8. The use of compound A: 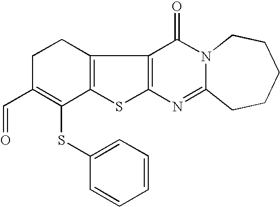
or a pharmaceutically acceptable salt thereof in the manufacture of a pharmaceutically acceptable preparation useful as 17β-hydroxysteroid dehydrogenase (17HSD) type 1 inhibitor.

9. The use of claim 8, characterized in that said pharmaceutical preparation is in the form of an oral formulation, injection, transdermal formulation or rectal formulation.

10. The use of claim 8, characterized in that said inhibitor is used for prevention or treatment of disorders caused by the 17HSD type 1 enzyme activity.

11. The use of claim 10, characterized in that said disorders comprise human breast cancer.

12. The use of compound A: 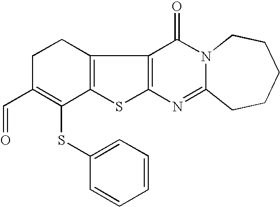
or a pharmaceutically acceptable salt thereof for prevention or treatment of disorders caused by the 17HSD type 1 enzyme activity.

13. The use of claim 12, characterized in that said disorders comprise human breast cancer.

14. A method for prevention or treatment of disorders caused by the 17HSD type 1 enzyme activity on a patient, characterized in that effective amount of compound A: 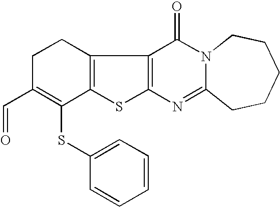
is administered to said patient.

15. The method of claim 14, characterized in that said disorder is human breast cancer.
